(19) United States
(12) Patent Application Publication
(10) Pub. No.: US 2013/0335233 A1
(43) Pub. Date: Dec. 19, 2013

(54) SYSTEMS AND METHODS FOR PORTABLE DEVICE COMMUNICATIONS AND INTERACTION

(71) Applicants: Anthony Kamar, Mesa, AZ (US); Kareem Mortada, Tempe, AZ (US)

(72) Inventors: Anthony Kamar, Mesa, AZ (US); Kareem Mortada, Tempe, AZ (US)

(21) Appl. No.: 13/915,616

(22) Filed: Jun. 11, 2013

Related U.S. Application Data
(60) Provisional application No. 61/658,288, filed on Jun. 11, 2012.

(51) Int. Cl. A61B 5/00; G01C 21/32

(52) U.S. Cl. CPC A61B 5/0002; G01C 21/32
USPC 340/870.01; 701/468; 701/454; 701/32.4

(57) ABSTRACT

In accordance with various aspects, a portable device tracking system can comprise a sensor device operatively coupled to a mobile object. The sensor device can comprises a microcontroller, a sensing unit coupled to the microcontroller, the sensing unit comprising a Global Positioning System (GPS) receiver to receive geographical data, and a transceiver coupled to the microcontroller. The portable device tracking system can also comprise a data manager configured to receive the geographical data from the sensor device. The sensor device can transmit data to the data manager across a network using a variety of communication protocols. Further, the portable device tracking system can be used to various applications, such as tracking an asset, a living object, a vehicle, or a medical device.

### CROSS-REFERENCE TO RELATED APPLICATIONS

This application claims priority to and benefit of U.S. Provisional Application No. 61/658,288, filed Jun. 11, 2012, and entitled “A PROCESS OF LOCATING OBJECTS THROUGH SMART PHONE APPLICATION”, and which is hereby incorporated by reference.

### FIELD OF INVENTION

The present invention relates to portable device tracking systems using various sensor devices.

### BACKGROUND OF THE INVENTION

Objects can often be misplaced or stolen, and some objects are worth tracking for this possibility. However, often the different objects don't have a built-in tracking system. Obviously a pet does not transmit its location, though it would be useful if the pet runs off. Moreover, certain objects, like vehicles and medical devices, can supply information worth tracking (in addition to tracking the location of the objects themselves). What is needed is a system for tracking such objects and information in a way that will not be disconnected and can be easily implemented.

### SUMMARY OF THE INVENTION

In accordance with various aspects, a portable device tracking system can comprise a sensor device operatively coupled to a mobile object. The sensor device can comprises a microcontroller, a sensing unit coupled to the microcontroller, the sensing unit comprising a Global Positioning System (GPS) receiver to receive geographical data, and a transceiver coupled to the microcontroller. The portable device tracking system can also comprise a data manager configured to receive the geographical data from the sensor device. In various embodiments, the mobile object may provide additional data to the sensor device, which can also be transmitted to the data manager.

The sensor device can transmit data to the data manager across a network using a variety of communication protocols. Further, the sensor device can transmit data in real-time, in batch form, or in predefined time intervals. In various embodiments, a user computing device, such as a computer or mobile phone, can access the data of the portable device tracking system. The information provided to the user computing device can include real-time data and historical data of a sensor device. Moreover, the portable device tracking system can be used in various applications, such as tracking an asset, a living object, a vehicle, or a medical device. For example, the sensor device can be attached to a coupled medical device via an I/O port of the medical device or wirelessly connect using one of protocols described herein. The sensor device can then transmit geographical data along with medical device data to the data manager. The advantages and implementation of the portable tracking system can vary by application as disclosed herein.

### BRIEF DESCRIPTION OF THE DRAWINGS

A more complete understanding of the embodiments of the present disclosure may be derived by referring to the detailed description at d claims when considered in connection with the following illustrative figures.

FIG. 1 illustrates a block diagram of an exemplary portable device tracking system;

FIG. 2 illustrates a block diagram of a sensor device in accordance with an exemplary embodiment;

FIG. 3 illustrates an object tracking application using an independent sensor device or a mobile phone in accordance with an exemplary embodiment;

FIG. 4A illustrates a vehicle tracking application using a sensor device in accordance with an exemplary embodiment;

FIG. 4B illustrates a vehicle tracking application using a Bluetooth-enable sensor device and mobile phone in accordance with an exemplary embodiment;

FIG. 4C illustrates a vehicle tracking application using a mobile phone in accordance with an exemplary embodiment; and

FIG. 5 illustrates a medical device tracking application using a sensor device and mobile phone in accordance with an exemplary embodiment.

### DETAILED DESCRIPTION OF EXEMPLARY EMBODIMENTS OF THE INVENTION

The present invention may be described herein in terms of various functional components and various processing steps. It should be appreciated that such functional components may be realized by any number of hardware or structural components configured to perform the specified functions.

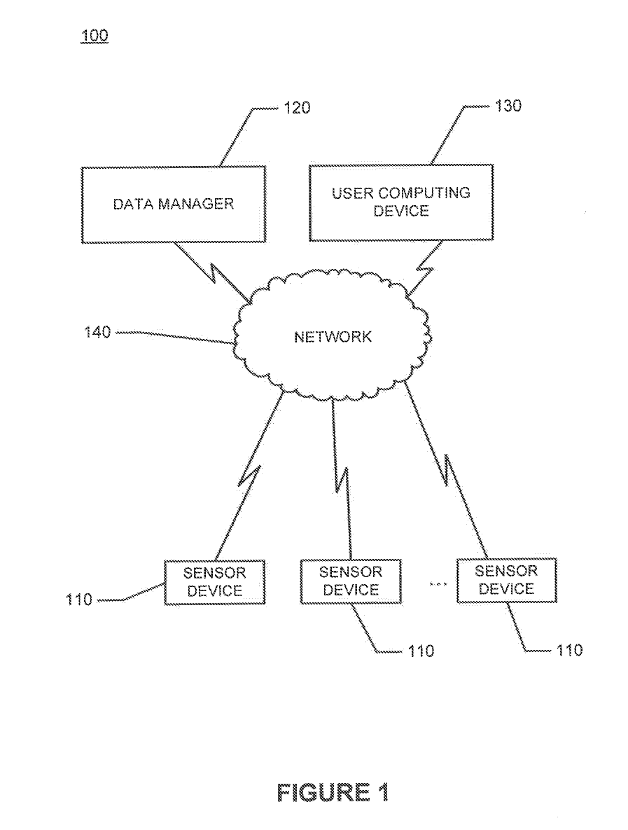

In accordance with various embodiments and with reference to FIG. 1, a portable device tracking system 100 can comprise a sensor device 110, a data manager 120, a user computing device 130, and a network 140. At a general level, sensor device 110 can communicate with data manager 120 over a variety of communication channels, such as GPS, satellite, cellular networks, wireless internet connection, Bluetooth, and the like. The data collected and received by the sensor device 110 can be communicated to the data manager 120, which can store the data in a cloud storage system or other server. The data manager 120 receives data from the portable sensor device 110 and can convert, filter, transform, and perform analysis on the data. The managed data is available for a customer via a user interface of user computing device 130.

The functionality of portable sensor device 110, data manager 120, computing device 130 and/or any other component operating in conjunction with aspects of the present invention can be implemented in any suitable manner, such as through a processor executing software instructions stored in a memory. Functionality may also be implemented through various hardware components storing machine-readable instructions, such as application-specific integrated circuits (ASICs), field-programmable gate arrays (FPGAs) and/or complex programmable logic devices (CPLDs).

### Sensor Device 110

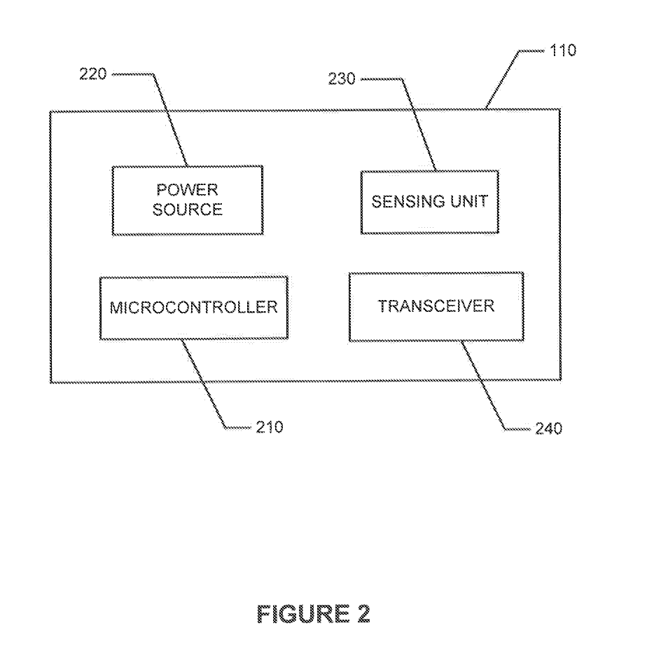

The sensor device 110 collects information regarding various coupled objects being monitored. An exemplary sensor device 110 is depicted in block diagram form in FIG. 2. In this exemplary embodiment, sensor device 110 includes a microcontroller 210, power source 220, sensing unit 230, and transceiver 240. As used herein, a “sensing unit” refers to any type of sensor, while a “sensor device” refers to any system or device capable of receiving data from one or more sensing units. As an example, a sensing unit may measure location, temperature, biometric parameters, or any object parameter, and the data is then received by sensor device 110.

### Microcontroller 210

In the exemplary sensor device 110 depicted in FIG. 2, microcontroller comprises a processor and memory. The processor retrieves and executes instructions stored in the memory to control the operation of the sensor device 110. Any number and type of microcontroller(s) such as an integrated circuit microprocessor, and/or digital signal processor (DSP), can be used in conjunction with the present invention. The microcontroller 210 may include, or operate in conjunction with, any other suitable components and features, such as comparators, analog-to-digital converters (ADCs), and/or digital-to-analog converters (DACs). The memory in microcontroller 210 can be capable of storing executable instructions, data, messages transmitted to or received from other components of system 100, and other information. A memory operating in conjunction with the present invention may include any combination of different memory storage devices, such as hard drives, random access memory (RAM), read only memory (ROM), FLASH memory, or any other type of volatile and/or nonvolatile memory.

In various embodiments, microcontroller 210 contains a controller algorithm that adapts sensor device operation based on current happenings. The sensor device 110 can enter a sleep mode when there is a low power level, or when the power level falls below a certain predefined threshold. Further, in various embodiments, sensor device 110 can enter a sleep mode if minimal change in data to be reported (e.g., not moving). The sensor device 110 can listen for an activation signal sent from the data manager 120 via the network 140. While in sleep mode, the sensor device 110 can continuously listen for the activation signal. In another embodiment, the sensor device 110 can listen for the activation signal during specific time intervals, thereby consuming less power.

### Power Source 220

The power source 22.0 powers the various components of the sensor device 110. The exemplary sensor device 110 depicted in FIG. 2 is powered by a solid-state Li-PON battery, though any number, combination, and type of suitable power sources can be utilized in embodiments of the present invention. In the exemplary sensor device 110 depicted in FIG. 2, the Li-PON battery is rechargeable via an external power connection. In various embodiments, the power source can include a solar power film.

### Sensor Unit 230

The sensor unit 230 measures characteristics related to a coupled object. The sensor unit 230 may be configured to measure any number of desired characteristics, such as location, temperature, biometric parameters, electrical parameters (such as voltage, resistance, and current), movement, and/or any other measurable characteristic. In various embodiments, sensor unit can comprise a UPS receiver, an accelerometer, and/or a thermocouple/thermistor. In various embodiments, sensor unit 230 further comprises an I/O data port for connecting to the coupled object. The coupled object may include its own sensors, or sensor data that can be transmitted by sensor device 110.

Information provided by the sensor unit 230 may be formatted as desired. For example, analog data regarding movement of a monitored object may be converted (using an analog to digital converter, for example) to a digital format, and subsequently formatted into a data packet including a data header followed by one or more data values. Similarly, the sensor device 110 may store a series of measurements from multiple sensor units 230 in the form of a table. Such tables can be transmitted remotely via network 140 to data manager 120, or accessed locally by a technician via a mobile device and a local wireless network.

### Transceiver 240

The transceiver 240 communicates with one or more other systems, such as the data manager 120, network 140, and/or any other suitable systems. Any suitable communications device, component, system, and method may be used in conjunction with the transceiver 240. In some exemplary embodiments, the transceiver 240 comprises a Bluetooth transceiver configured to communicate with a data manager 120.

The sensor device 210 may include, or operate in conjunction with, any type and number of transceivers 240. In some embodiments, the sensor device 110 includes a cellular radio frequency (RF) transceiver and may be configured to communicate using any number and type of cellular protocols, such as General Packet Radio Service (GPRS), Global System for Mobile Communications (GSM), Enhanced Data rates for GSM Evolution (EDGE), Personal Communication Service (PCS), Advanced Mobile Phone System (AMPS), Code Division Multiple Access (CDMA), Wideband CDMA (W-CDMA) Time Division-Synchronous CDMA (TD-SCDMA), Universal Mobile Telecommunications System (UMTS), and/or Time Division Multiple Access (TDMA). The transceiver 240 may communicate using any other wireless protocols, such as a Zigbee protocol, a Wibree protocol, an IEEE 802.11 protocol, an IEEE 802.15 protocol, an IEEE 802.16 protocol, an Ultra-Wideband (UWB) protocol, an Infrared Data Association (IrDA) protocol, a Bluetooth protocol, and combinations thereof.

A sensor device 110 operating in conjunction with the present invention may alternatively (or additionally) communicate using any other method of wired or wireless communication. For example, in some embodiments the transceiver 240 may be configured to communicate using one or more wired connections using, without limitation: tip and sleeve (TS), tip, ring, and sleeve (TRS), and tip, ring, ring, and sleeve (TRRS) connections; serial peripheral interface bus (SPI) connections; universal serial bus (USB) connections; RS-232 serial connections, Ethernet connections, optical fiber connections, and Firewire connections. The transceiver 240 can be configured (e.g., through a software program residing in microcontroller 210) to detect and switch to different communication protocols and/or different wired or wireless connections, thus allowing communications with a wide variety of devices.

The sensor device 110 communicates with other systems (such as data manager 120 and user computing device 130) via network 140. In some embodiments, such as in the exemplary system 100 depicted in FIG. 1, sensor device 110 is disposed within communication range of at least one network 140. In some embodiments, sensor device 110 communicates with network 140 using a wired or wireless communication protocol (e.g., a longer-range protocol such as a cellular protocol), including those described previously.

### Network 140

The network 140 allows the sensor devices 110 to communicate with other systems and devices, such as data manager 120 and user computing device 130. The network 140 may include any combination of wired and wireless connections and protocols, such as those described above. The network 140 may comprise a local area network (LAN), wide area network (WAN), wireless mobile telephony network, General Packet Radio Service (GPRS) network, wireless Local Area Network (WLAN), Global System for Mobile Communications (GSM) network, Personal Communication Service (PCS) network, Advanced Mobile Phone System (AMPS) network, and/or a satellite communication network. In some embodiments, network 140 includes the Internet to allow the data manager 120 or computing device 130 to communicate with sensor devices 110 from anywhere an Internet connection can be established. As such, embodiments of the invention provide efficient, centralized monitoring of assets even in applications (such as oil and gas production) where monitored assets are in remote locations and often spread across large areas.

### Data Manager 120

In the exemplary embodiment depicted in FIG. 1, the data manager 120 receives and analyzes data from the sensor devices 110 and can issue commands to control sensor device 110 and/or an asset being monitored. In various embodiments, the data manager 120 communicates with one or more sensor devices 110. The data manager 120 may be configured to communicate using any desired wired or wireless communication connection or protocol, including those described above. In some embodiments, the data manager 120 is configured to communicate with a plurality of sensor devices 110 and, in turn, communicate with other data managers 120 via the network 140. In this manner, a single data manager can communicate with multiple sensor devices 110 using a longer-range protocol.

The data manager 120 may receive data from the sensor devices 110 in any desired manner. In some embodiments, the data manager 120 is configured to automatically request data from one or more of the sensor devices 110 via the network 140. Alternatively, the sensor device 110 or any other device operating in conjunction with embodiments of the invention can be configured to automatically request and/or transmit data in any suitable manner. For example, each sensor device 110 may be configured to collect and send data measured from a monitored object (such as an asset or pet) and automatically transmit such data to the data manager 120 at periodic intervals (e.g., every 15 seconds or every 5 minutes or any other time interval) via network 140.

The transmission of data by a sensor device operating in conjunction with the present invention may be subject to any suitable conditions or rules that dictate whether the data is transmitted. For example, a sensor device may first check to verify (1) that it is within range of a network; (2) that the sensor device has sufficient battery reserves to send the request and transmit the data; (3) that the data manager is ready to receive the data, and/or whether any other suitable condition is met.

### User Computing Device 130

A user computing device 130 can communicate with any of the other components in system 100. The user computing device 130 may include a personal computer or a mobile computing device, such as a laptop computer, a mobile wireless telephone, a smartphone, tablet computer, a personal digital assistant (PDA), or any other computer-based system with a visual display.

A user can use computing device 130 to view, in real-time or near-real-time, the status of any of the components of a system of the present invention, such as the components shown in the Figures. The computing device 130 may also be used to send commands to control such components or to the monitored asset, as well as to view reports showing data from the sensor devices 110, or to analyze the data to generate metrics regarding the status of the monitored asset. Data can be provided to or received from a user of the computing device 130 in a machine-readable format. The computing device 130 may be configured to send, receive, and process machine-readable data can in any standard format (such as a MS Word document, Adobe PDF file, ASCII text file, JPEG, or other standard format) as well as any proprietary format. Machine-readable data to or from the user interface may also be encrypted to protect the data from unintended recipients and/or improper use.

The data manager 120 or user computing device 130 may include any number and type of processors to retrieve and execute instructions stored in the memory storage device of the server to control its functionality. The data manager 120 may include any type of conventional computer, computer system, computer network, computer workstation, minicomputer, mainframe computer, or computer processor, such as an integrated circuit microprocessor or microcontroller in accordance with the present invention. The data manager 120 or computing device 130 operating in conjunction with the present invention may include any combination of different memory storage devices, such as hard drives, random access memory (RAM), read only memory (ROM), FLASH memory, or any other type of volatile and/or nonvolatile memory. The data manager 120 may include an operating system (e.g., Windows, OS2, UNIX, Linux, Solaris, MacOS, etc.) as well as various conventional support software and drivers typically associated with computers. Software applications stored in the memory may be entirely or partially served or executed by the processor(s) in performing methods or processes of the present invention.

The data manager 120 or computing device 130 may also include a user interface for receiving and providing data to one or more users. The user interface may include any number of input devices such as a keyboard, mouse, touch pad, touch screen, alphanumeric keypad, voice recognition system, or other input device to allow a user to provide instructions and information to other components in a system of the present invention. Similarly, the user interface may include any number of suitable output devices, such as a monitor, speaker, printer, or other device for providing information to one or more users.

Any of the components can be configured to communicate with each other (or with other additional systems and devices) for any desired purpose. For example, the data manager 120 or user computing device 130 may be used to upload software to sensor device 110 or other component, provide or update encryption keys, and to perform diagnostics on any of the components in system 100. Any computer system may be configured (i.e., using appropriate security protocols) to communicate instructions, software upgrades, data, and other information with components via network 140. In some embodiments, data received from the sensor devices 110 is processed into a report and electronically provided (i.e., via email) to multiple users in a ubiquitous data format such as Portable Document Format (PDF). Such reports can be created at the request of a user or generated automatically at predetermined times or in response to the occurrence of an event (such as a detected problem with a monitored asset).

Any combination and/or subset of the elements of the methods depicted herein may be practiced in any suitable order and in conjunction with any system, device, and/or process. The method described herein can be implemented in any suitable manner, such as through software operating on one or more systems or devices, including the system 100.

### Transmit Data

Data collected from a sensor device 110 or generated by any other device operating in conjunction with the present invention may be transmitted to other systems, such as to data manager 120 for collection and/or analysis. The data can be transmitted in any suitable manner, including using any of the wired or wireless communication methods and protocols described previously. Any amount of data can be transmitted in any manner. For example, data from the sensor device 110 can be transmitted as it is measured, or data can be stored (such as in a memory storage device in the sensor device 110) for a period of time before being transmitted to another device, in some cases, for example, it may be more efficient to transmit blocks of data at once rather than initiating communication with another device each time data is available. In other cases, a device may be out of range or otherwise unavailable to receive the data. The data can also be stored for any desired length of time, and/or until a particular event occurs. For example, the device data could be stored until it is verified that the receiving data manager 120 have received the data, allowing the data to be retransmitted if necessary. Data can also be deleted when a data record exceeds a predetermined storage time, and/or the oldest data record is deleted first after a predetermined storage size limit has been reached.

Data transmitted from the sensor devices 110 may be validated to ensure it was transmitted properly and completely. The sensor device data may also be validated to ensure it was provided from a specific sensor device 110 or group of sensor devices 110 (i.e., associated with a particular object being monitored). The data may also be validated to ensure that fields in the data correspond to predetermined values and/or are within certain thresholds or tolerances. Any number, code, value or identifier can be used in conjunction with validating the device data. For example, the data can be validated by analyzing a serial number, a device identifier, one or more parity bits, a cyclic redundancy checking code, an error correction code, and/or any other suitable feature.

In exemplary embodiments of the present invention, various components (such as data manager 120) may be configured to receive data directly or indirectly from a sensor device 110, format a message based on the data, and transmit the formatted message to another system or device. This functionality may be implemented through software operating on any suitable mobile computing device and with any computer operating system.

### Commands from Data Manager

In addition to receiving and processing data from the sensor devices 110 and other components operating in conjunction with embodiments of the invention, the data manager 120 (or user computing device 130 if desired) can transmit a command to control various functions of such components, the object being monitored, or other systems and devices. Any number of commands of any type may be transmitted by the data manager 120 to any suitable recipient. The command can be transmitted using the same variety of wired and wireless methods discussed previously. For example, the data manager 120 may issue a command to control, reconfigure, and/or update a software application operating on the sensor device 110.

By way of example only, three applications of the system and sensor device are described herein. The invention is in no way intended to be limited to the three described applications, but merely offered for clarification of the invention.

### Application to Track Objects

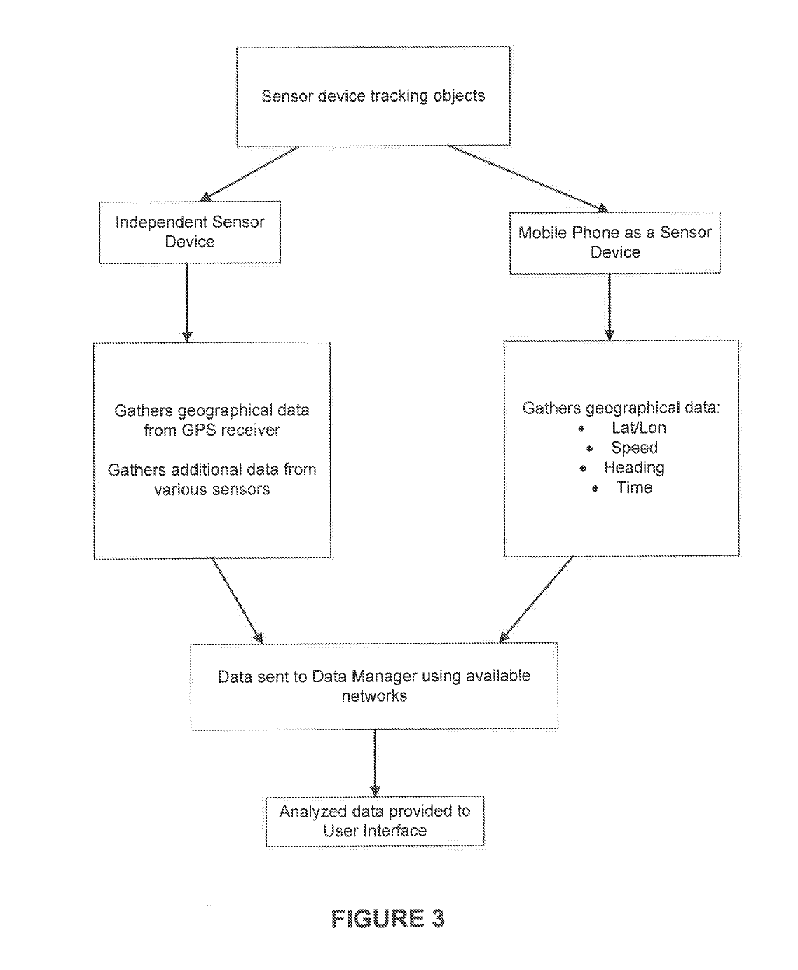

In accordance with various embodiments and with reference to FIG. 3, one application of the portable device tracking system 100 is to track objects such as assets, a living object (people, pets, etc.) and other mobile objects. A GPS receiver, in a sensor device coupled to the mobile object, receives information from multiple satellites, and the location information of the mobile object is transmitted using a wireless transceiver as described above. The data manager can receive the GPS information from the sensor device, and convert the GPS information into location data readable by the user computing device. In various embodiments, the GPS information can be translated into geographical coordinates and show the location on a map on the user computing device. The location data can be useful for theft protection and/or misplacement of the mobile objects.

One example of a mobile object to track is a mobile phone. The mobile phone can include software and hardware to gather geographical data such as latitude/longitude coordinates, speed, heading, time, and other data collected by a mobile phone. The gathered geographical data can be sent through the network (using GSM or RF for example) to the data manager.

Another example is a sensor device that can be attached to a mobile object such as a pet, child, package, equipment, and the like. The sensor device can be attached by a clip, adhesive, plug-in, fasteners, contained within the mobile object, and other ways of attachment as would be known by one skilled in the art. The GPS receiver in the sensor device can receive the location data as mentioned above, which is then transmitted by the sensor device to the data manager. As with tracking a mobile phone, the location data and other information can be displayed to a user computing device on a user interface. The user interface can display not only the real-time location of the mobile object, but also the past locations of the mobile object. A user interface map can show the progression of the mobile object over a past time frame as selected by a user, such as the past 8 hours or one week or any other time interval. The location information can be useful for verifying the mobile object is at an appropriate destination, and also show the route by which the mobile object arrived at the appropriate destination.

### Application to Track One or More Vehicles

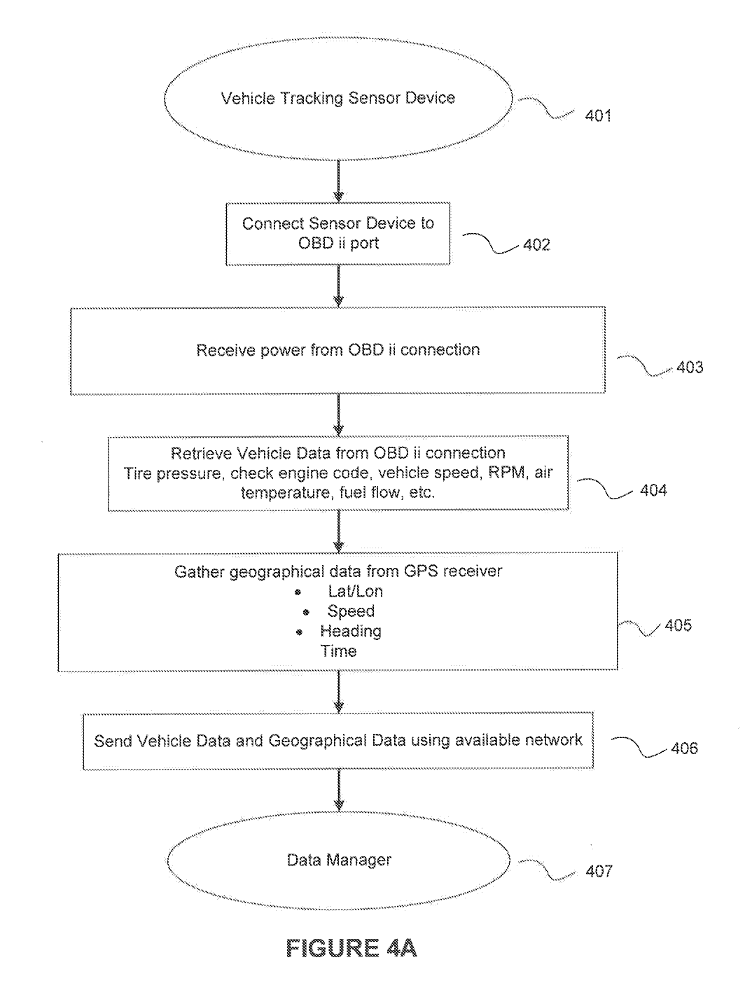

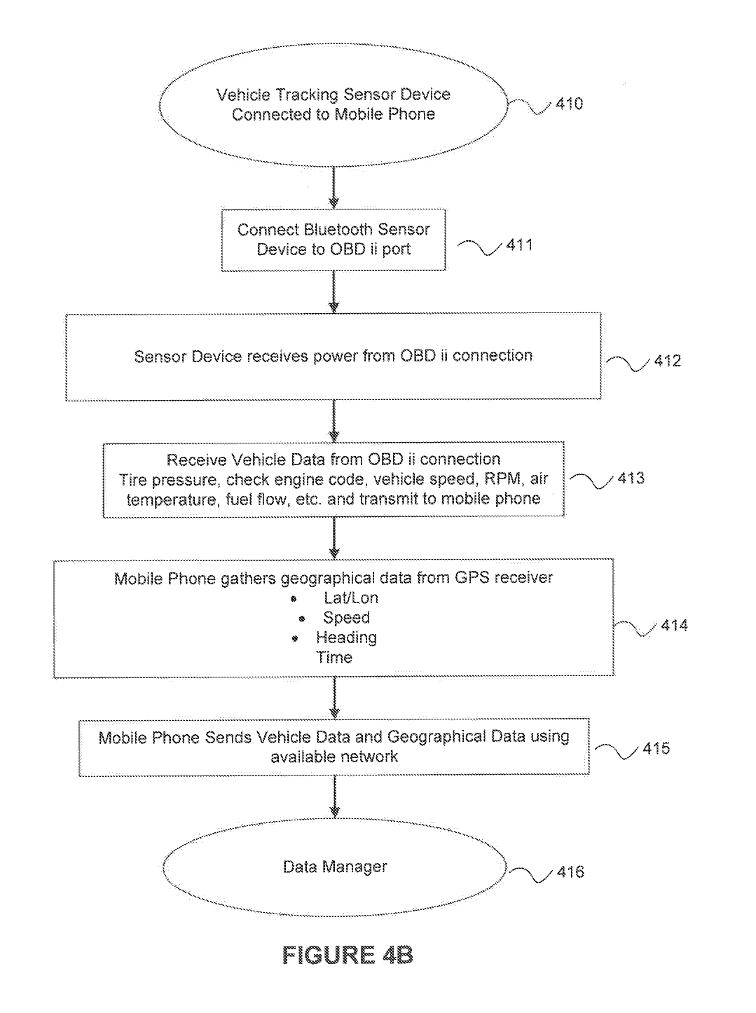

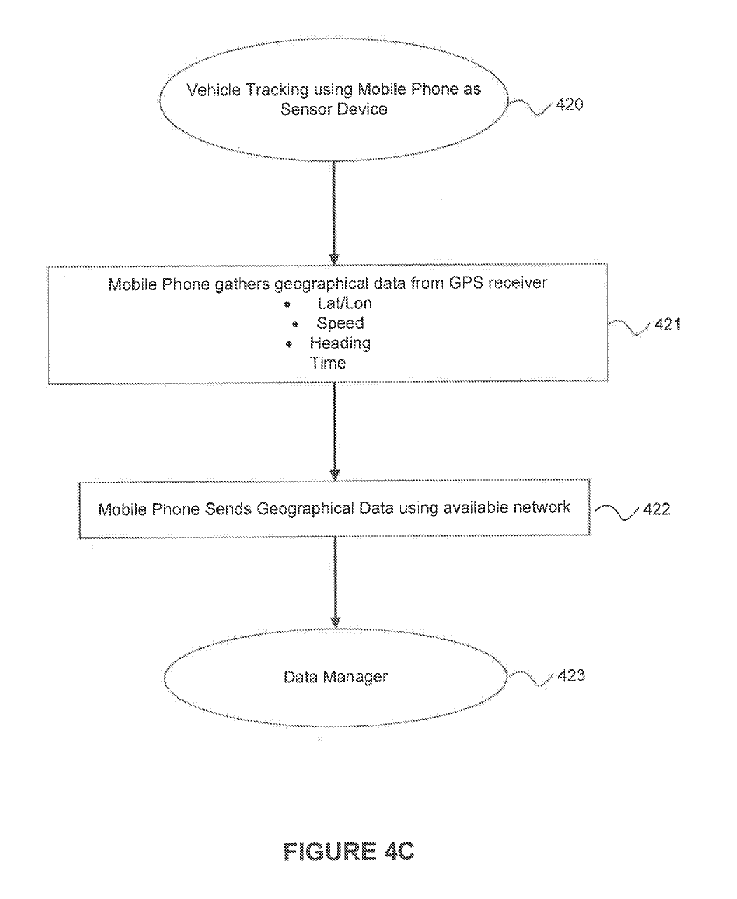

In accordance with various embodiments, a second application of the portable device tracking system 100 is to track one or more vehicles and/or vehicle data. In accordance with various embodiments and with reference to FIG. 4A, a vehicle tracking sensor device 401 can be connected to an OBD ii (On Board Diagnostics) port on the vehicle 402. The sensor device can receive power from the OBD ii port 403. Additionally, the sensor device can be programmed to retrieve vehicle data from the OBD ii connection 404. The vehicle data can include, for example, tire pressure, a check engine code, vehicle speed, RPMs, air temperature, fuel flow, and the readings of various sensors (for example, oxygen sensors). Each vehicle manufacturer can have different data available via an OBD ii connection, and the sensor device program can be modified accordingly. Furthermore, the sensor device can still have its own GPS receiver, and still provide data on latitude/longitude coordinates, heading, speed, and time 405. The sensor device can be configured to transmit both the vehicle data and the geographical data using the sensor device transceiver to the data manager 407 via an available network 406. Furthermore, commands can be sent to the sensor device from the data manager or user computing device to control the data transmission (type, frequency) as further described herein.

In various embodiments and with reference to FIG. 4B, a vehicle tracking Bluetooth-enable sensor device 410 can be connected to OBD ii port 411. Similar to the above, the sensor device can receive power from the OBD ii port 412. Furthermore, the sensor device can be programmed to retrieve vehicle data from the OBD ii connection and be paired with a mobile phone for transmitting the vehicle data 413. Moreover, the sensor device does not have a GPS receiver. However, the local smartphone can be used to gather the geographical data from an internal GPS receiver 414. The local smart phone then transmits the data to the data manager 416 via the network (in this case, a GSM network or an RF network if available) 415. Furthermore, commands can he sent to the sensor device from the data manager or user computing device to control the data transmission (e.g., type, frequency), and commands to the device to control the smart phone power performance (again by changing transmit rates).

Similar to the two vehicle tracking embodiments previously described and with reference to FIG. 4C, the vehicle tracking can also be accomplished by using a mobile phone as the sensor device 420. The mobile phone can be used to gather the geographical data from an internal GPS receiver 421. The mobile phone then transmits the data to the data manager 423 via the network (in this case, a GSM network or an RF network if available) 422. This embodiment does not provide diagnostic information of the vehicle, but can still track the vehicle's location, speed, and direction.

In addition, all of the vehicle tracking embodiment can be extended to tracking a fleet of vehicles, with each vehicle having its own sensor device. The fleet tracking application can benefit a company trying to monitor fleet location, such as a taxi cab company, or a company trying to having more efficient routing by analyzing historical route data, such as a delivery company.

### Application to Track Medical Devices

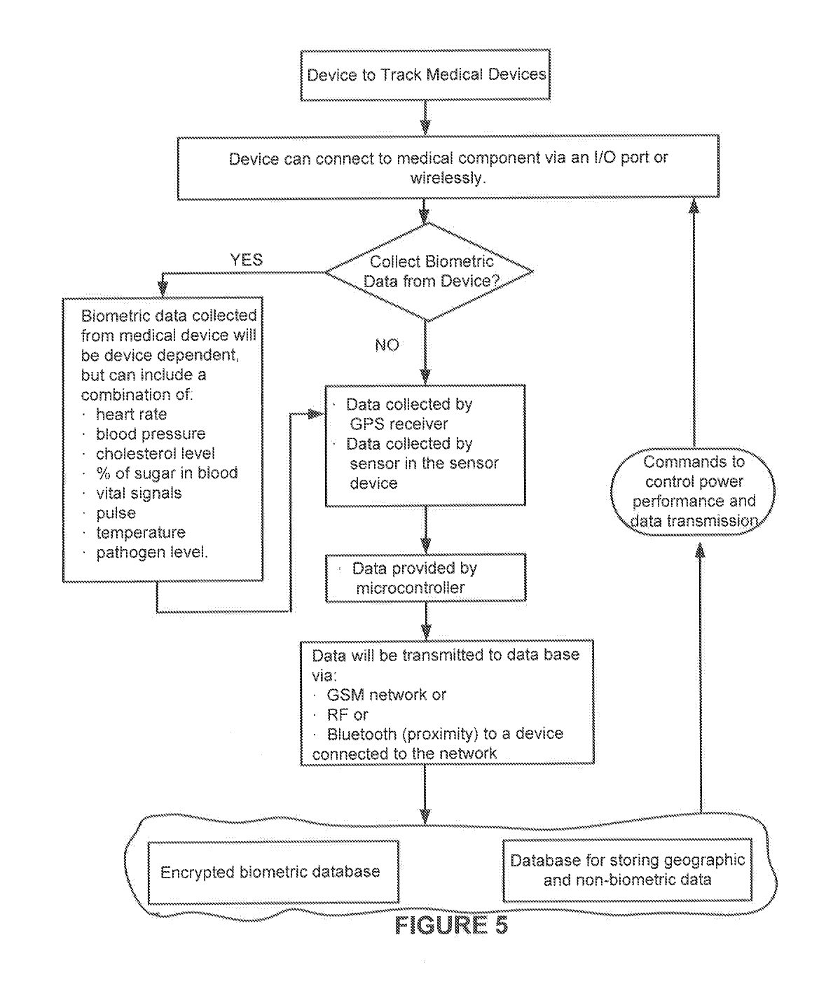

In accordance with various embodiments and with reference to FIG. 5, a third application of the portable device tracking system 100 is to track a medical device and/or information supplied by a medical device. The sensor device can be attached to a coupled medical device via an I/O port of the medical device or wirelessly connect using one of protocols described herein. The sensor device can draw power from the I/O port connection if a wired connection or be battery powered if a wireless connection, and data can also be transferred from the coupled medical device to the sensor device. The sensor device can include the GPS receiver, which receives latitude/longitude coordinates and time stamp. Further, the sensor device can receive non-biometric data from the coupled medical device, such as humidity, ambient temperature, and any other signal that may affect patient hostility. In various embodiments, the sensor device can also relay information about the sensor device itself, such as power level and health of the sensor device.

In various embodiments, the sensor device may also receive biometric data from the coupled medical device. The type of biometric data collected from the coupled medical device is medical device dependent. However, as an example, the biometric data could include one or more of heart rate, blood pressure, cholesterol level, blood sugar levels, vital signs, temperature, pathogen levels, and the like.

The collected data can be transmitted by the sensor device to the data manager via the network using any of the above disclosed protocols, specifically including a GSM network, an RF network, or a wireless proximity connection such as Bluetooth. An advantage of the sensor device being coupled to the medical device is the ability to provide real-time updates of the data collected by the medical device. Current medical devices already being used may collect the biometric data and then a healthcare provider may later download the biometric data once a patient is at a healthcare facility. By coupling the sensor device to the medical device, the biometric data can be more readily available and monitored. The sensor device can be an economical solution to upgrade a standard medical device to a real-time wirelessly monitoring medical device.

The data manager can receive the data and store the data in one of two database types. A first database type can store biometric data and any data related to a patient. The first database type can be an encrypted database for medical personnel use and complies with United States laws, such as Health Insurance Portability and Accountability Act (HIPAA). The second database type can store all remaining data, such as geographical data, sensor device parameters, and sensor device environment data. Access to the first and second database types can be limited to authorized users, and specifically to certain records in the databases. For example, only an authorized healthcare provider can have access to the biometric database, and all access can be restricted to patients of said healthcare provider. However, other users can be able to access the non-biometric database for monitoring of the medical device. For example, an administrator may access the database to determine where a medical device is located in a facility, or if the medical device has been transferred to another location. Furthermore, an insurance company may access the non-biometric database to view the location history of a medical device if a claim has been filed. The added advantage of being able to view the historical location data may help an insurance company determine whether the limitations and restrictions on an insurance policy were met or violated.

In all the applications described herein and as generally disclosed, the data manager can transmit a command to the sensor device to control power performance and data transmission rates by updating the sensor device's firmware. The sensor device can be instructed to transmit data at a slower time interval, or not at all, if the sensor device is in the same location for set period of time. Drawing on the example above, if the sensor device is monitoring a vehicle and the vehicle has been in the same location for 15 minutes, the sensor device can be set to not transmit data again until the sensor device has moved ten feet or more, or some other predefined distance. Moreover, the sensor device can be set to store the recorded data and transmit the recorded data in batch form. Further, the batch transmissions can be set to occur during off-peak hours for reduced cost. Reducing the transmission rate saves both on the power used by the sensor device, and also on the cost of data transmission over the network.

Once the data manager has received data from the sensor device, the data manager can analyze the data. The data manager can also filter or summarize the received data to provide a subset of data to the user computing device. In various embodiments, user settings can also be applied to set the filters. For example, a user can select a timeframe of data such as 8 hours or one week to view. The user can also select to only see data points spaced every 5 minutes or 30 minutes or any other time interval, rather than all the data points received from the sensor device.

Moreover, the data manager can compare the received geographical data from sensor device to prior geographical data. In one embodiment, if the geographical coordinates are the same between the current and prior data, then the data manager may discard the data. In this way, the data manager stores less data in a database and only movement, instead of multiple database entries showing the mobile object has not changed location. Furthermore, in another embodiment, the data manager can indicate that a mobile object has been halted in response to the mobile object's geographical coordinates not changing over a predetermined time period, like 15 minutes for example.

An additional use of the data received from the sensor device is to use the data to target the user with geo-targeted advertising and marketing. The portable device tracking system tracks the mobile object and notes the location and duration spent at that location. This information can be used to determine the consumer habits of a person carrying the sensor device. The data can be used to provide a user or the sensor device carrier advertising based on user behaviors. In one embodiment, the geo-targeted advertising can be provided to the user in real-time while using the user computing device and tracking an object. In another embodiment, the geo-targeted advertising can be provided to the user based on the historical patterns and routes of the mobile object for later use.

### User Interface of User Computing Device

In accordance with various embodiments, the tracking data can be presented to a user using a web-based software program available on the user computing device. The software program can be a package of different modules, which increases the flexibility of the software program. In various embodiments, the user interface is customizable per application. The user interface features and functionality can be set by a data manager programmer or by a user. Since the software program is modular, the user can make different selections for features, and the user interface program rebuilds per user's customization. Accordingly, the software program is designed to allow a user to add or remove features as desired and the user interface be automatically rebuilt per the changes in real-time or near real-time. Furthermore, in various embodiments, the portable device tracking system and user interface is can be a subscription-based program, and the different feature selections can result in different pricing for the system usage. Examples of the features include reporting and analysis. If a user initially only wanted data analysis to be presented on the user interface, the user would be charged a first price. If the user selected to add reporting capabilities at a later time, then the user would be charged a second price, higher than first price. Advantageously, the user can switch between the various features as needed, and the data manager tracks which features are available and sets the prices accordingly.

Additional features of the user interface include the capability to disable a select sensor device. This may be one of many sensor devices for a user or the user's only sensor device. In response to the sensor device being disabled on the user interface, a disable signal is sent from the data manager to the selected sensor device via the network. The sensor device is then disabled from transmitted data back to the data manager. However, the sensor device can still be powered and receiving information. Another feature of the user interface is the capability to enable a select disabled sensor device. Similar to disabling, an enable signal is sent to the disabled sensor device from the data manager, thereby enabling the sensor device to start transmitting data.

Another feature of the user interface is to provide labels for certain geographical coordinates. For example, specific locations can be labeled by a user, such as home, office, school, Company A, Company B, and the like. This feature can help a user quickly determine a mobile object's location.

Systems and devices operating in accordance with aspects of the present invention may implement one or more security measures to protect data, restrict access, or provide any other desired security feature. For example, any device operating in conjunction with the present invention may encrypt transmitted data and/or protect data stored within the device itself. Such security measures may be implemented using hardware, software, or a combination thereof. Any method of data encryption or protection may be utilized in conjunction with the present invention, such as public/private keyed encryption systems, data scrambling methods, hardware and software firewalls, tamper-resistant or tamper-responsive memory storage devices or any other method or technique for protecting data. Similarly, passwords, biometrics, access cards or other hardware, or any other system, device, and/or method may be employed to restrict access to any device operating in conjunction with the present invention.

The present invention has been described above with reference to various exemplary embodiments. However, those skilled in the art will recognize that changes and modifications may be made to the exemplary embodiments without departing from the scope of the present invention. These alternatives can be suitably selected depending upon the particular application or in consideration of any number of factors associated with the operation of the system. Moreover, these and other changes or modifications are intended to be included within the scope of the present invention, as expressed in the following claims.

What is claimed is:

1. A portable device tracking system comprising:
a sensor device operatively coupled to a mobile object, wherein the sensor device comprises:
a microcontroller;
a sensing unit coupled to the microcontroller, the sensing unit comprising a Global Positioning System (GPS) receiver to receive geographical data;
a transceiver coupled to the microcontroller;
a data manager configured to receive the geographical data from the sensor device.

2. The system of claim 1, wherein the sensor device further comprises a power source.

3. The system of claim 1 wherein the transceiver is configured to transmit data using one or more of: a Zigbee protocol, a Wibree protocol, an IEEE 802.11 protocol, an IEEE 802.15 protocol, an IEEE 802.16 protocol, an Ultra-Wideband (UWB) protocol, an Infrared Data Association (IrDA) protocol, a Bluetooth protocol, and combinations thereof.

4. The system of claim 1, wherein the transceiver transmits data to the data manager through a network.

5. The system of claim 4, wherein the network comprises one or more of a local area network (LAN), wide area network (WAN), wireless mobile telephony network, General Packet Radio Service (GPRS) network, wireless Local Area Network (WLAN), Global System for Mobile Communications (GSM) network, Personal Communication Service (PCS) network, Advanced Mobile Phone System (AMPS) network, a satellite communication network, and combinations thereof.

6. The system of claim 1, wherein the sensor device is configured to transmit the data intermittently to the data manager.

7. The system of claim 1, wherein the mobile object is a living object.

8. The system of claim 7, wherein the data manager transmits the geographical data to as user computing device for displaying on a user interface.

9. The system of claim 8, wherein the user interface displays the real-time location of the living object on a map.

10. The system of claim 8, wherein the user interface displays the past locations of the living object on a map.

11. The system of claim 1, wherein the mobile object is a vehicle.

12. The system of claim 11, wherein the sensor device is connected to an OBD ii port, and wherein the sensor device retrieves vehicle data from the OBD ii connection.

13. The system of claim 12, wherein the sensor device further transmits the vehicle data to the data manager.

14. The system of claim 12, wherein the sensor device further transmits the vehicle data to a mobile phone, and wherein the mobile phone transmits geographical data and the vehicle data to the data manager.

15. The system of claim 11, wherein the sensor device is a mobile phone, and wherein the mobile phone transmits geographical data to the data manager.

16. The system of claim 1, wherein the mobile object is a medical device, and wherein the sensor device is coupled to an I/O port of the medical device.

17. The system of claim 16, wherein the medical device communicates medical device data to the sensor device, and wherein the medical device data includes at least one of biometric data and non-biometric data.

18. The system of claim 17, wherein the biometric data comprises one or more of heart rate, blood pressure, cholesterol level, blood sugar levels, vital signs, temperature, pathogen levels, and combinations thereof.

19. The system of claim 17, wherein the sensor device transmits the medical device data in real-time.

20. The system of claim 17, wherein the data manager receives the medical device data, and wherein the data manager stores the medical device data in either a HIPAA compliant database in response to receiving biometric data, or stores in a standard database in response to receiving non-biometric data.
